(19) United States
(12) Patent Application Publication
(10) Pub. No.: US 2025/0051436 A1
(43) Pub. Date: Feb. 13, 2025

(54) Arthritis Treatment

(71) Applicant: XBIOTECH INC., Vancouver (CA)

(72) Inventor: John Simard, Austin, TX (US)

(21) Appl. No.: 18/824,526

(22) Filed: Sep. 4, 2024

Related U.S. Application Data
(63) Continuation of application No. 17/807,939, filed on Jun. 21, 2022, now Pat. No. 12,116,405.
Application No. 17/807,939 is a continuation of application No. 16/530,667, filed on Aug. 2, 2019, now Pat. No. 11,390,672.
Application No. 16/530,667 is a continuation of application No. 15/235,263, filed on Aug. 12, 2016, now abandoned.
Application No. 15/235,263 is a continuation of application No. 13/162,705, filed on Jun. 17, 2011, now abandoned.
(60) Provisional application No. 61/356,176, filed on Jun. 18, 2010.

(51) Int. Cl. C07K 16/24; A61K 39/00; A61K 39/395; A61P 27/02; A61P 29/00

(52) U.S. Cl. CPC C07K 16/245; A61K 39/39533; A61K 2039/505; A61P 27/02; A61P 29/00

(57) ABSTRACT

Administration of a monoclonal Ab (mAb) that specifically targets IL-1α is useful to treating articular and extra-articular symptoms of arthritis.

### CROSS-REFERENCE TO RELATED APPLICATIONS

This application is a continuation application of U.S. patent application Ser. No. 17/807,939 filed on Jun. 21, 2022, which is a continuation application of U.S. patent application Ser. No. 16/530,667 filed on Aug. 2, 2019 (now U.S. Pat. No. 11,390,672), which is a continuation of U.S. patent application Ser. No. 15/235,263 filed on Aug. 12, 2016 (now abandoned), which is a continuation application of U.S. patent application Ser. No. 13/162,705 filed on Jun. 17, 2011 (now abandoned), which claims priority from U.S. provisional patent application Ser. No. 61/356,176 filed on Jun. 18, 2010. All patent applications are incorporated herein by reference in their entirety.

### STATEMENT AS TO FEDERALLY SPONSORED RESEARCH

Not applicable.

### FIELD OF THE INVENTION

The invention relates generally to the fields of immunology, inflammation, arthritis, and medicine. More particularly, the invention relates to the use of antibodies (Abs) which specifically bind interleukin-1α (IL-1α) to treat one or more symptoms of arthritis.

### BACKGROUND

Arthritis, the most common cause of disability in the United States, is a collection of different conditions such as osteoarthritis, rheumatoid arthritis, gout, psoriatic arthritis, septic arthritis, and reactive arthritis. All types of arthritis are characterized by joint inflammation which causes pain, swelling, redness, stiffness, and warmth at that affected site. Because afflicted subjects are less mobile due to pain and stiffness, arthritis can indirectly lead to obesity, high cholesterol, and/or heart disease. Arthritis can also cause extra-articular disease such as iritis, uveitis, oral ulcers, inflammation of the gastrointestinal tract, inflammation of the genitourinary tract, and skin lesions.

For most types of arthritis, no cure exists and treatment is largely symptomatic, e.g., administration of analgesics and anti-inflammatory drugs. Non-steroidal anti-inflammatory drugs (NSAIDs) can be used to reduce inflammation and pain. While generally effective, NSAIDs may cause side effects such as abdominal pain, bleeding, ulcers, and liver and kidney damage. Corticosteroids are effective at reducing inflammation and joint damage, but can cause a number of side effect are also associated including bruising, weight gain, cataracts, bone thinning, diabetes, and hypertension. Other drugs commonly used to treat arthritis are methotrexate, cyclosporine, cyclophosphamide, leflunomide, hydroxychloroquine, sulfasalazine, and minocycline. These too can cause side effects such as liver damage and immunosuppression. Tumor necrosis factor (TNF) inhibitors like etanercept (Enbrel), infliximab (Remicade), and adalimumab (Humira) are also useful for treating arthritis. Side effects of TNF inhibitors include injection site reactions, heart failure, lymphoma, and increased risk of infection.

### SUMMARY

The invention is based on the discovery that administration of an antibody (Ab) that specifically targets IL-1α in a human subject suffering from arthritis reduces the number of CD14+IL-1α+ peripheral blood monocytes in the subject and markedly ameliorates inflammation in both articular and extraarticular sites—all without any observed side effects other than pain at the administration site.

Accordingly, the invention features a method of treating an inflammatory pathology associated with arthritis in a human subject by administering to the subject a pharmaceutical composition including a pharmaceutically acceptable carrier and an amount of an anti-IL-1α antibody effective to reduce at least one symptom of the inflammatory pathology in the subject. The symptom can be joint inflammation such as of the wrist or shoulder, or inflammation of the eye such as uveitis. The anti-IL-1α antibody can be a monoclonal antibody such as an IgG1. The anti-IL-1α antibody can be the monoclonal antibody designated as MABp1 or a monoclonal antibody that includes one or more complementarity determining regions (CDRs) of MABp1.

The pharmaceutical composition can be administered to the subject by injection, subcutaneously, intravenously, intramuscularly, intraocularly, or directly into an inflamed joint. The antibody might also be administered to the eye topically. In the method, the amount of the anti-IL-1α antibody effective to reduce at least one symptom of the inflammatory pathology in the subject can be sufficient to raise the subject's peripheral blood concentration of anti-IL-1α antibody to at least 4 ug/ml; and/or sufficient to decrease the number of the subject's CD14+IL-1a+ peripheral blood monocytes by at least 5%.

The method might also include a step of measuring the number of CD14+IL-1α+ monocytes in the subject's peripheral blood after administration of the pharmaceutical composition, e.g., wherein the step of measuring the number of CD14+IL-1α+ monocytes in the subject's peripheral blood is performed at least two different time points after administration of the pharmaceutical composition.

In another aspect, the invention features a method inducing monocyte vacuolization in a subject by administering to the subject a pharmaceutical composition including a pharmaceutically acceptable carrier and an amount of an anti-IL-1α antibody effective to induce vacuole formation in monocytes.

Unless otherwise defined, all technical terms used herein have the same meaning as commonly understood by one of ordinary skill in the art to which this invention belongs. Commonly understood definitions of biological terms can be found in Rieger et al., Glossary of Genetics: Classical and Molecular, 5th edition, Springer-Verlag: New York, 1991; and Lewin, Genes V, Oxford University Press: New York, 1994. Commonly understood definitions of medical terms can be found in Stedman's Medical Dictionary, 27^{th} Edition, Lippincott, Williams & Wilkins, 2000.

As used herein, an “antibody” or “Ab” is an immunoglobulin (Ig), a solution of identical or heterogeneous Igs, or a mixture of Igs. An “antibody” can also refer to fragments and engineered versions of Igs such as Fab, Fab′, and F(ab′)₂ fragments; and scFv's, heteroconjugate Abs, and similar artificial molecules that employ Ig-derived CDRs to impart antigen specificity. A “monoclonal antibody” or “mAb” is an Ab expressed by one clonal B cell line or a population of Ab molecules that contains only one species of an antigen binding site capable of immunoreacting with a particular epitope of a particular antigen. A “polyclonal antibody” or “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

When referring to a protein molecule such as an Ab, “purified” means separated from components that naturally accompany such molecules. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. Purity can be measured by any appropriate method, e.g., column chromatography, polyacrylamide gel electrophoresis, or HPLC analysis. A chemically-synthesized protein or other recombinant protein produced in a cell type other than the cell type in which it naturally occurs is “purified.”

By “bind”, “binds”, or “reacts with” is meant that one molecule recognizes and adheres to a particular second molecule in a sample, but does not substantially recognize or adhere to other molecules in the sample. Generally, an Ab that “specifically binds” another molecule has a K_{d} greater than about 10⁵, 10⁶, 10⁷, 10⁸, 10⁹, 10¹⁰, 10¹¹, or 10¹² liters/mole for that other molecule.

A “therapeutically effective amount” is an amount which is capable of producing a medically desirable effect in a treated animal or human (e.g., amelioration or prevention of a disease or symptom of a disease).

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

FIG. 1 is a graph and table showing the pharmacokinetics of MABp1 after administration to a human subject with reactive arthritis.

FIGS. 2A, 2B, and 2C show a series of graphs and histograms showing flow cytometric blood analyses after administration of MABp1 to a human subject with reactive arthritis.

FIG. 3 is a series of graphs showing flow cytometric blood analyses after administration of MABp1 to a human subject with reactive arthritis.

### DETAILED DESCRIPTION

The invention encompasses compositions and methods for treating a symptom or pathologic process associated with arthritis in a subject. The below described preferred embodiments illustrate adaptation of these compositions and methods. Nonetheless, from the description of these embodiments, other aspects of the invention can be made and/or practiced based on the description provided below.

### General Methodology

Methods involving conventional immunological and molecular biological techniques are described herein. Immunological methods (for example, assays for detection and localization of antigen-Ab complexes, immunoprecipitation, immunoblotting, and the like) are generally known in the art and described in methodology treatises such as Current Protocols in Immunology, Coligan et al., ed., John Wiley & Sons, New York. Techniques of molecular biology are described in detail in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York. Ab methods are described in Handbook of Therapeutic Abs, Dubel, S., ed., Wiley-VCH, 2007. General methods of medical treatment are described in McPhee and Papadakis, Current Medical Diagnosis and Treatment 2010, 49^{th} Edition, McGraw-Hill Medical, 2010; and Fauci et al., Harrison's Principles of Internal Medicine, 17^{th} Edition, McGraw-Hill Professional, 2008

### Treatment of Arthritis Symptoms

The compositions and methods described herein are useful for treating an inflammatory pathology associated with arthritis in a mammalian subject by administering to the subject a pharmaceutical composition including an amount of an anti-IL-1α antibody effective to reduce at least one symptom of the inflammatory pathology in the subject. The mammalian subject might be any that suffers from arthritis including, human beings, dogs, cats, horses, cattle, sheep, goats, and pigs. Human subjects might be male, female, adults, children, seniors (65 and older), and those with other diseases. The particular symptom or pathologic process associated with arthritis can be inflammation, pain, stiffness, or degeneration of a joint (e.g., in the wrist, digits [metacarpal or metatarsal joints], elbows, shoulders, hips, knees, ankles, foot, neck, or back) or extraarticular tissue (e.g., iritis, uveitis, oral ulcers, inflammation of the gastrointestinal tract, inflammation of the genitourinary tract, or skin lesions).

Antibodies and Other Agents that Target IL-1α

Any suitable type of Ab that specifically binds IL-1α and reduces a symptom or pathologic process caused by arthritis in a subject might be used in the invention. For example, the anti-IL-1α Ab used might be mAb, a polyclonal Ab, a mixture of mAbs, or an Ab fragment or engineered Ab-like molecule such as an scFv. The Ka of the Ab is preferably at least 1×10⁹ M⁻¹ or greater (e.g., greater than 9×10¹⁰ M⁻¹, 8×10¹⁰ M⁻¹, 7×10¹⁰ M⁻¹, 6×10¹⁰ M⁻¹, 5×10¹⁰ M⁻¹, 4×10¹⁰ M⁻¹, 3×10¹⁰ M⁻¹, 2×10¹⁰ M⁻¹, or 1×10¹⁰ M⁻¹). In a preferred embodiment, the invention utilizes a fully human mAb that includes (i) an antigen-binding variable region that exhibits very high binding affinity for human IL-1α and (ii) a constant region that is effective at both activating the complement system though C1q binding and binding to several different Fc receptors. The human Ab is preferably an IgG1, although it might be of a different isotype such as IgM, IgA, or IgE, or subclass such as IgG2, IgG3, or IgG4. One example of a particularly useful mAb is MABp1, an IL-1α-specific IgG1 monoclonal antibody described in U.S. patent application Ser. No. 12/455,458 filed on Jun. 1, 2009. Other useful mAbs are those than include at least one but preferably all the CDRs of MABp1.

Because B lymphocytes which express Ig specific for human IL-1α occur naturally in human beings, a presently preferred method for raising mAbs is to first isolate such a B lymphocyte from a subject and then immortalize it so that it can be continuously replicated in culture. Subjects lacking large numbers of naturally occurring B lymphocytes which express Ig specific for human IL-1α may be immunized with one or more human IL-1α antigens to increase the number of such B lymphocytes. Human mAbs are prepared by immortalizing a human Ab secreting cell (e.g., a human plasma cell). See, e.g., U.S. Pat. No. 4,634,664.

In an exemplary method, one or more (e.g., 5, 10, 25, 50, 100, 1000, or more) human subjects are screened for the presence of such human IL-1α-specific Ab in their blood. Those subjects that express the desired Ab can then be used as B lymphocyte donors. In one possible method, peripheral blood is obtained from a human donor that possesses B lymphocytes that express human IL-1α-specific Ab. Such B lymphocytes are then isolated from the blood sample, e.g., by cells sorting (e.g., fluorescence activated cell sorting, “FACS”; or magnetic bead cell sorting) to select B lymphocytes expressing human IL-1α-specific Ig. These cells can then be immortalized by viral transformation (e.g., using EBV) or by fusion to another immortalized cell such as a human myeloma according to known techniques. The B lymphocytes within this population that express Ig specific for human IL-1α can then be isolated by limiting dilution methods (e.g., cells in wells of a microtiter plate that are positive for Ig specific for human IL-1α are selected and subcultured, and the process repeated until a desired clonal line can be isolated). See, e.g., Goding, Monoclonal Abs: Principles and Practice, pp. 59-103, Academic Press, 1986. Those clonal cell lines that express Ig having at least nanomolar or picomolar binding affinities for human IL-1α are preferred. MAbs secreted by these clonal cell lines can be purified from the culture medium or a bodily fluid (e.g., ascites) by conventional Ig purification procedures such as salt cuts, size exclusion, ion exchange separation, and affinity chromatography.

Although immortalized B lymphocytes might be used in in vitro cultures to directly produce mAbs, in certain cases it might be desirable to use heterologous expression systems to produce mAbs. See, e.g., the methods described in U.S. patent application Ser. No. 11/754,899. For example, the genes encoding an mAb specific for human IL-1α might be cloned and introduced into an expression vector (e.g., a plasmid-based expression vector) for expression in a heterologous host cell (e.g., CHO cells, COS cells, myeloma cells, and E. coli cells). Because Igs include heavy (H) and light (L) chains in an H₂L₂ configuration, the genes encoding each may be separately isolated and expressed in different vectors.

Although generally less preferred due to the greater likelihood that a subject will develop an anti-Ab response, chimeric mAbs (e.g., “humanized” mAbs), which are antigen-binding molecules having different portions derived from different animal species (e.g., variable region of a mouse Ig fused to the constant region of a human Ig), might be used in the invention. Such chimeric Abs can be prepared by methods known in the art. See, e.g., Morrison et al., Proc. Nat'l. Acad. Sci. USA, 81:6851, 1984; Neuberger et al., Nature, 312:604, 1984; Takeda et al., Nature, 314:452, 1984. Similarly, Abs can be humanized by methods known in the art. For example, monoclonal Abs with a desired binding specificity can be humanized by various vendors or as described in U.S. Pat. Nos. 5,693,762; 5,530,101; or 5,585,089.

The mAbs described herein might be affinity matured to enhance or otherwise alter their binding specificity by known methods such as VH and VL domain shuffling (Marks et al. Bio/Technology 10:779-783, 1992), random mutagenesis of the hypervariable regions (HVRs) and/or framework residues (Barbas et al. Proc Nat. Acad. Sci. USA 91:3809-3813, 1994; Schier et al. Gene 169:147-155, 1995; Yelton et al. J. Immunol. 155:1994-2004, 1995; Jackson et al., J. Immunol. 154(7):3310-9, 1995; and Hawkins et al, J. Mol. Biol. 226:889-896, 1992. Amino acid sequence variants of an Ab may be prepared by introducing appropriate changes into the nucleotide sequence encoding the Ab. In addition, modifications to nucleic acid sequences encoding mAbs might be altered (e.g., without changing the amino acid sequence of the mAb) for enhancing production of the mAb in certain expression systems (e.g., intron elimination and/or codon optimization for a given expression system). The mAbs described herein can also be modified by conjugation to another protein (e.g., another mAb) or non-protein molecule. For example, a mAb might be conjugated to a water soluble polymer such as polyethylene glycol or a carbon nanotube (See, e.g., Kam et al., Proc. Natl. Acad. Sci. USA 102: 11600-11605, 2005). See, U.S. patent application Ser. No. 11/754,899.

Preferably, to ensure that high titers of human IL-1α-specific mAb can be administered to a subject with minimal adverse effects, the mAb compositions of the invention are at least 0.5, 1, 2, 3, 4, 5, 6, 7, 8, 9, 10, 11, 12, 13, 14, 15, 20, 25, 30, 35, 40, 45, 50, 60, 70, 80, 90, 95, 96, 97, 98, 99, 99.9 or more percent by weight pure (excluding any excipients). The mAb compositions of the invention might include only a single type of mAb (i.e., one produced from a single clonal B lymphocyte line) or might include a mixture of two or more (e.g., 2, 3, 4, 5, 6, 7, 8, 9, 10 or more) different types of mAbs.

To modify or enhance their function, the human IL-1α mAbs might be conjugated another molecule such as a cytotoxin. A human IL-1α specific mAb might be conjugated with one or more cytotoxins to more effectively kill cells expressing IL-1α. Cytotoxins for use in the invention can be any cytotoxic agent (e.g., molecule that can kill a cell after contacting the cell) that can be conjugated to a human IL-1α specific mAb. Examples of cytotoxins include, without limitation, radionuclides (e.g., ³⁵S, ¹⁴C, ³²P, ¹²⁵I, ¹³¹I, ⁹⁰Y, ⁸⁹Zr, ²⁰¹Tl, ¹⁸⁶Re, ¹⁸⁸Re, ⁵⁷Cu, ²¹³Bi, and ²¹¹At), conjugated radionuclides, and chemotherapeutic agents. Further examples of cytotoxins include, but are not limited to, antimetabolites (e.g., 5-fluorouricil (5-FU), methotrexate (MTX), fludarabine, etc.), anti-microtubule agents (e.g., vincristine, vinblastine, colchicine, taxanes (such as paclitaxel and docetaxel), etc.), alkylating agents (e.g., cyclophasphamide, melphalan, bischloroethylnitrosurea (BCNU), etc.), platinum agents (e.g., cisplatin (also termed cDDP), carboplatin, oxaliplatin, JM-216, CI-973, etc.), anthracyclines (e.g., doxorubicin, daunorubicin, etc.), antibiotic agents (e.g., mitomycin-C), topoisomerase inhibitors (e.g., etoposide, tenoposide, and camptothecins), or other cytotoxic agents such as ricin, diptheria toxin (DT), Pseudomonas exotoxin (PE) A, PE40, abrin, saporin, pokeweed viral protein, ethidium bromide, glucocorticoid, anthrax toxin and others. See, e.g., U.S. Pat. No. 5,932,188.

While the IL-1α specific Abs described above are preferred for use the invention, in some cases, other agents that specifically target IL-1α might be used so long as their administration leads to improvement of one or more symptoms of arthritis. These other agents might include small organic molecules, aptamers, peptides, and proteins that specifically bind IL-1α.

### Pharmaceutical Compositions and Methods

The anti-IL-1α Ab compositions may be administered to animals or humans in pharmaceutically acceptable carriers (e.g., sterile saline), that are selected on the basis of mode and route of administration and standard pharmaceutical practice. A list of pharmaceutically acceptable carriers, as well as pharmaceutical formulations, can be found in Remington's Pharmaceutical Sciences, a standard text in this field, and in USP/NF. Other substances may be added to the compositions and other steps taken to stabilize and/or preserve the compositions, and/or to facilitate their administration to a subject.

For example, the Ab compositions might be lyophilized (see Draber et al., J. Immunol. Methods. 181:37, 1995; and PCT/US90/01383); dissolved in a solution including sodium and chloride ions; dissolved in a solution including one or more stabilizing agents such as albumin, glucose, maltose, sucrose, sorbitol, polyethylene glycol, and glycine; filtered (e.g., using a 0.45 and/or 0.2 micron filter); contacted with beta-propiolactone; and/or dissolved in a solution including a microbicide (e.g., a detergent, an organic solvent, and a mixture of a detergent and organic solvent.

The Ab compositions may be administered to animals or humans by any suitable technique. Typically, such administration will be parenteral (e.g., intravenous, subcutaneous, intramuscular, or intraperitoneal introduction). The compositions may also be administered directly to the target site (e.g., an inflamed joint, or the uvea or conjuctiva) by, for example, injection or topical application. Other methods of delivery, e.g., liposomal delivery or diffusion from a device impregnated with the composition, are known in the art. The composition may be administered in a single bolus, multiple injections, or by continuous infusion (e.g., intravenously or by peritoneal dialysis).

A therapeutically effective amount is an amount which is capable of producing a medically desirable result in a treated animal or human. An effective amount of anti-IL-1α Ab compositions is an amount which shows clinical efficacy in arthritis patients as measured by the improvement in pain and function as well as the prevention of structural damage. As is well known in the medical arts, dosage for any one animal or human depends on many factors, including the subject's size, body surface area, age, the particular composition to be administered, sex, time and route of administration, general health, and other drugs being administered concurrently. A preferred dose is one that is sufficient to raise the subject's peripheral blood concentration of anti-IL-1α Ab to at least 4 (e.g., at least 4, 5, 6, 7, 8, 9, 10, 20, 30, 40, 50, 100, 200, 300, 400, 500, 1000, 2500, or 5000) micrograms/ml. It is expected that an appropriate dosage of Abs would be in the range of about 0.2 to 20 (e.g., 0.5, 1, 2, 3, 4, 5, 6, 7, 8, 9, 10, 15, 20, 30, 50, or 100) mg/kg body weight for subcutaneous administration and about 0.001 to 50 (e.g., 0.001, 0.01, 1, 5, 10, 15, 25, or 50) mg per eye for topical administration to the eye. The dose may be given repeatedly, e.g., hourly, daily, weekly, or monthly.

### EXAMPLES

### Example 1—Xilonix™

Xilonix™ is a sterile injectable liquid formulation of 15 mg/mL MABp1 in a stabilizing isotonic buffer (pH 6.4). Each 10-mL Type I borosilicate glass serum vial contains 5 mL of the formulation, and is sealed with a 20-mm Daikyo Flurotec butyl rubber stopper and flip-off aluminum seal. The product is stored at 5±3° C., with excursions to room temperature permitted. The exact composition of the drug product is shown below:

Composition of the Drug Product (Xilonix ™)

| Ingredients | Grade | Manufacturer | Concentration |
|---|---|---|---|
| MABp1 antibody | GMP | XBiotech | 15 mg/mL |
| sodium phosphate dibasic | compendial | JT Baker | 12 mg/mL |
| citric acid monohydrate | compendial | JT Baker | 2 mg/mL |
| Trehalose•2H2O (high- | compendial | Ferro- | 60 mg/mL |
| purity low endotoxin) | | Pfanstiehl | |
| polysorbate 80 | compendial | JT Baker | 0.2 mg/mL |
| Phosphoric acid, to | compendial | JT Baker | 0.04 mg/mL |
| adjust pH | | | |
| water for injection | compendial | Microbix | q.s. |

### Example 2—Treatment of Reactive Arthritis with an IL-1α-Specific Monoclonal Antibody

A 48 year-old male patient with reactive arthritis was administered a total 220 milligrams of MABp1, an IL-1α-specific monoclonal antibody described in U.S. patent application Ser. No. 12/455,458 filed on Jun. 1, 2009. The patient had a long history of reactive arthritis, starting at age 16, when he was diagnosed with Reiter's syndrome during hospitalization for severe inflammation in his left knee. This inflammation resolved, yet the patient experienced periodic relapses in several joints until his mid-twenties. No further episodes occurred until, at age 35, the patient had a severe unilateral episode of uveitis that lasted for 8 weeks. The uveitis was poorly managed with ophthalmic corticosteroids and oral NSAIDS, resulting in some scaring. The patient subsequently experienced at least three additional episodes of uveitis of varying intensities, one episode requiring subcorneal injection of corticosteroids.

Just prior to his 48^{th} birthday, the patient developed severe pain in his left shoulder and wrist. Evident swelling and redness with almost complete loss of mobility affected the wrist. The patient was unable to abduct his left arm greater than about 20° due to intense shoulder pain. On that day, the patient was given a subacromial injection of corticosteroids into the left shoulder. The patient reported that the condition continued to worsen with pain from shoulder and wrist reportedly becoming continuous, interrupting work and preventing sleep. In addition, pain and irritation in the left eye ensued, indicating onset of an episode of uveitis. This was reportedly the first time joint inflammation and uveitis occurred together. The patient was taking ophthalmic corticosteroids, oral and topical ophthalmic NSAIDS with little apparent benefit.

On day 0 (forty-two days after the subacromial injection of corticosteroids), the patient was administered four subcutaneous injections of MABp1, delivering a total of 110 mg of MABp1 (in equal doses). No side effects other than pain during injection was reported. Blood was drawn by venous puncture immediately prior to injection into two 5 ml sodium heparin tubes. Plasma analysis using an enzyme-linked immunadsorbant assay (ELISA) for the detection of existing endogenous anti-IL-1α antibodies revealed no pre-existing antibodies.

On day 1, the patient reported that he woke up that morning without the throbbing pain that had become the “first sensation upon waking.” Over the next several days there was an evident improvement in mobility. There was no induration or redness at the injection sites. A blood draw was taken and flow cytometric analysis (FACS) was performed to evaluate leukocyte subsets and IL-1α expression on monocytes. Analysis was also performed on plasma to determine levels of MABp1 and to begin collection of pharmacokinetic (pK) data for MABp1. FACS analysis of PBMC revealed that most CD14+ monocytes (72.6%) expressed IL-1α. A MABp1 plasma concentration of 3.2 μg/ml was observed.

On day 6, another blood sample was taken and analyzed using FACS and for MABp1. The frequency of CD14+ monocytes stained by MABp1 had declined to 47.3%. Plasma levels of MABp1 had increased to 7 μg/ml. Although not confirmed, the increase in MABp1 concentration was considered to reflect a depot effect of the subcutaneous administration of MABp1. Although there had been improvement, the patient still exhibited considerable tenderness and pain with movement and the uveitis had flared since the previous weekend, where the patient had attended a party and consumed alcohol. The patient was administered another 110 mg of MABp1 subcutaneously.

On day 14, a blood sample was taken and analyzed using FACS and pK analysis was performed on plasma. CD14+ monocyte frequency stained by MABp1 further declined to 21.7%. However, plasma levels of MABp1 had also declined to 5.8 μg/ml. This was unanticipated, since plasma levels of MABp1 had increased over the week after the first injection.

Approximately one month after the first injection of MABp1 the patient was reevaluated. Marked improvement was noted in mobility and there was no pain in the wrist. Pain in shoulder was present only upon abduction to 90°. FACS analysis revealed no detectable CD14+ monocytes stained by MABp1. Plasma levels of MABp1 had declined to 1.6 μg/ml, suggesting a half-life for MABp1 of about two weeks.

Over the course of the next several weeks the patient showed gradual but continuous improvement in mobility. There was complete resolution of the uveitis. The improvement was noted even though the patient discontinued use of all medications after the first injection of MABp1. Approximately three months after the first injection of MABp1, the frequency of CD14+ monocytes stained by MABp1 had returned to pre-treatment levels. MABp1 levels in plasma declined to 0.07 μg/ml. However, the patient continued to do well with continuing improvement in mobility of the shoulder.

### Example 3—Screening of Plasma Samples for Endogenous Autoantibody Against hIL-1A and Pharmacokinetics of MABp1

A method was developed for the screening of plasma samples for endogenous autoantibody against human IL-1α (hIL-1α) using a direct ELISA. This method was also used to determine pharmacokinetics (pK) of MABp1 after administration, with the exception, that higher dilutions plasma samples were made.

The direct ELISA involves coating of recombinant human IL-1α on a polystyrene microplate. The bound human IL-1A captures endogenous anti-human IL-1α antibody from test samples. An HRP-conjugated-Fc specific, mouse-anti-human IgG is then used to detect the captured endogenous anti-human IL-1A antibody, followed by treatment with TMB substrate. On reacting with HRP enzyme, the TMB substrate produces a deep blue-colored soluble product. The enzymatic reaction is stopped by the addition of a stop solution that turns the blue-colored product to yellow. The colorimetric measurements are carried out on a microplate reader at 450 nm.

About 5 ml plasma sample per sample is provided. Plasma is kept at 2-8° C. prior to aliquoting and storage at −80° C. Plasma samples are diluted 1:500, 1:1000 and 1:2000-fold to use as samples. A positive control in buffer is used containing 20 μg/ml MABp1 antibody stock as 1:5,000 and 1:10,000-fold dilutions on microplate. Buffer is used as a negative control as well as a pre-determined negative control plasma, which is diluted as 1:1,000, 1:2,000 and 1:5,000. An additional positive plasma control is used, which is plasma spiked with 20 μg/ml MABp1 antibody and diluted as 1:5,000 and 1:10,000 for samples on the microplate.

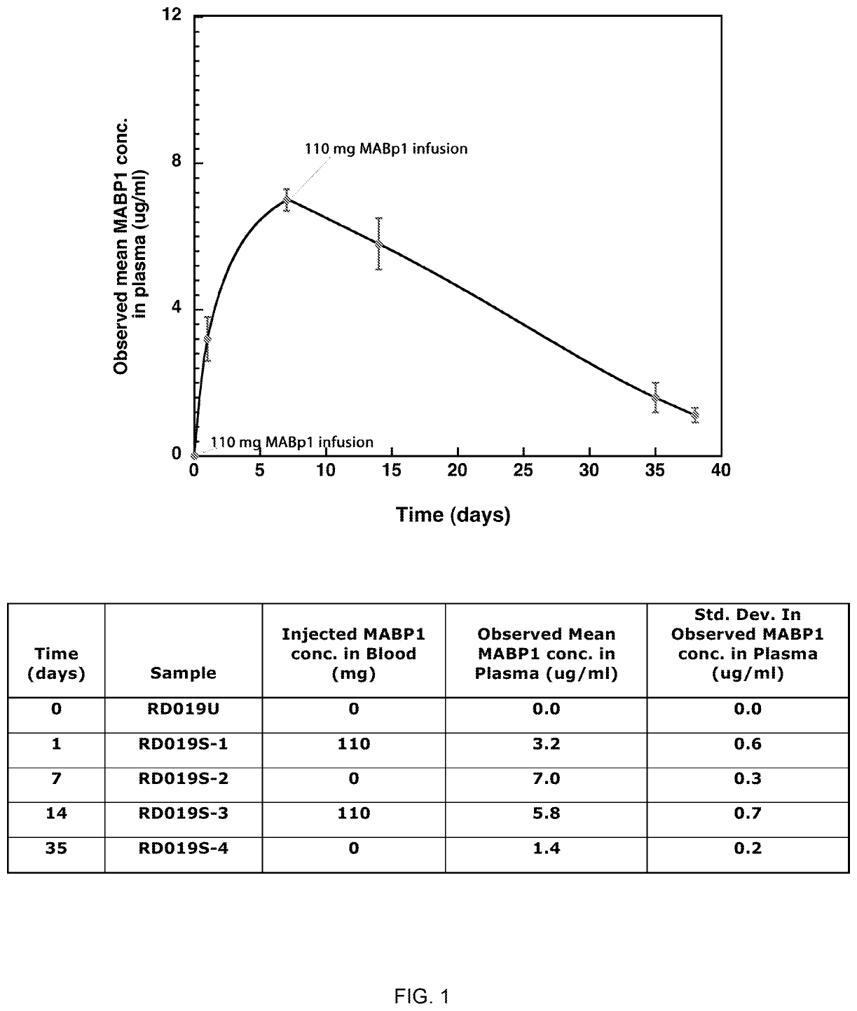

If the positive control value falls within ±2 standard deviation, the ELISA data is considered acceptable. However if the QC positive control value falls beyond ±2 standard deviation, the ELISA data is considered unacceptable and the experiment would be repeated. Using a Kaleidagraph, the logarithmic mean absorbance of standard solution is plotted as a function of logarithmic concentration along with absorbance error bars. The standard curve should exhibit a linear behavior. Results from a pharmacokinetics analysis of samples taken from the patient as described in Example 2 are shown in FIG. 1.

### Example 4—Flow Cytometric (FACS) Examination of Blood Lineage Subsets

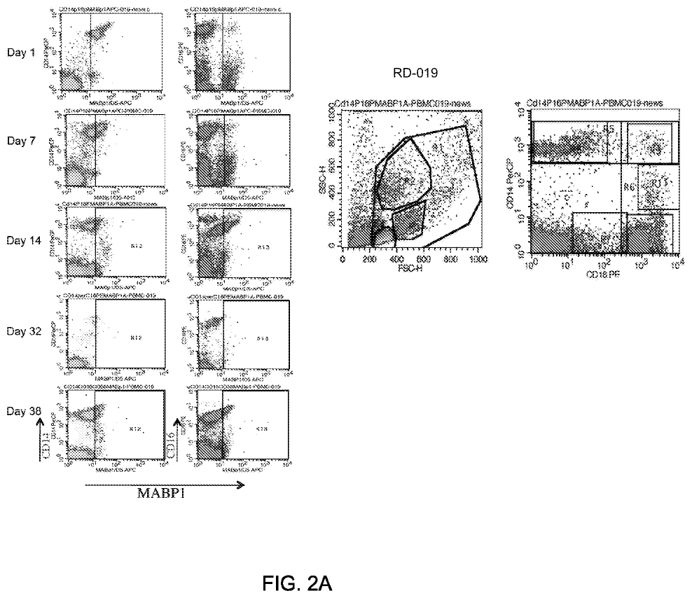

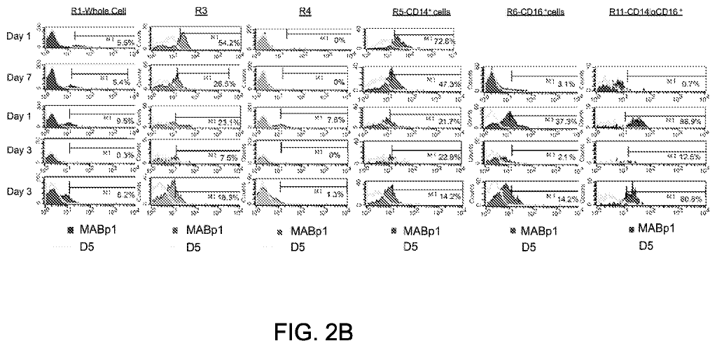

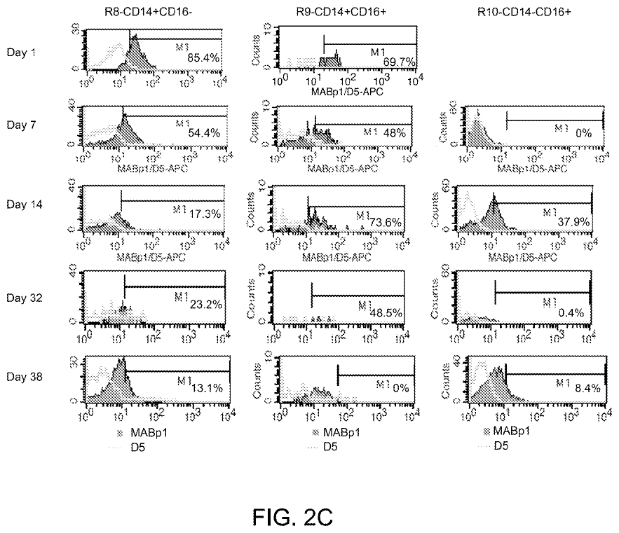

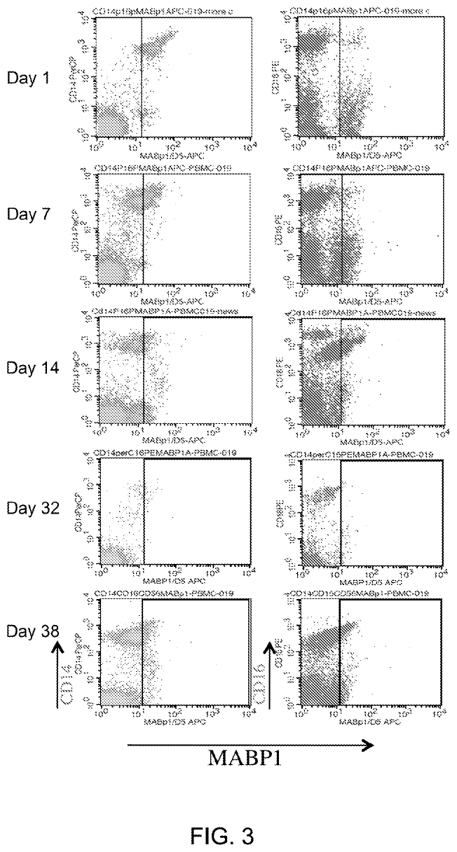

FACS procedures are described for both whole blood staining, and staining of peripheral blood mononuclear cells (PBMC) enriched from whole blood. Both whole blood and PBMC staining was performed on all samples. This FACS analysis allows relative percentage determination of blood lineage subsets: B and T lymphocytes, NK cells, monocytes, neutrophils, and IL-1α+ cells. Results from FACs analyses of samples taken from the patient as described in Example 2 are shown in FIGS. 2 and 3. A photomicrograph of a blood smear showed that MABp1 administration caused extensive vacuolization in peripheral blood monocytes when analyzed 32 days post administration.

### Example 5—Treatment of Uveitis with an IL-1α-Specific Monoclonal Antibody

About two months following resolution of the uveitis described in Example 2, the patient experienced another episode of uveitis (predominantly iritis). The patient was started on corticosteroid and non-steroidal anti-inflammatory drops (NSAIDS). Oral NSAIDS were also used. The uveitis was unresponsive to treatment and progressed. However, there was no evidence of any joint involvement, with shoulder continuing to show improvement in mobility. The patient was administered MABp1 topically to the affected eye. MABp1 (15 mg/ml solution) was administered at a rate of one drop per minute, for ten minutes, for a total of ten drops to the affected eye (approximately 3.75 mg in 0.25 ml). The patient did not complain of any pain during the administration. However, for several hours after, the patient reported discomfort and burning. Oral NSAIDs were taken and the patient slept. The next morning, the patient reported considerable improvement, reduced pain and less inflammation than prior to administration. Twenty-four hours after the first administration of the MABP1 drops, the patient administered 10 drops in the same fashion. Again, discomfort and burning was noted. Oral NSAIDs were taken, and again the patient took bed rest. The uveitis resolved itself completely. No further medications were taken. No recurrence of uveitis was observed over the next four months.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating joint inflammation caused by arthritis in a human subject, the method comprising the step of administering to the human subject a pharmaceutical composition comprising a pharmaceutically acceptable carrier and an anti-IL-1α antibody that binds human IL1α with the Kₐ of at least 1×10⁹ M⁻¹ to at a dosage of 0.2 to 20 mg/kg body weight, wherein the human subject has reduced joint inflammation associated with the arthritis after the treatment.

2. The method of claim 1, wherein the anti-IL-1α antibody is a monoclonal antibody.

3. The method of claim 2, wherein the monoclonal antibody is an IgG1.

4. The method of claim 1, wherein the pharmaceutical composition is administered to the subject more than once.

5. The method of claim 1, wherein the pharmaceutical composition is administered to the subject subcutaneously.
